(19) United States
(12) Patent Application Publication
(10) Pub. No.: US 2021/0244205 A1
(43) Pub. Date: Aug. 12, 2021

(54) FOOT MIRROR ASSEMBLY

(71) Applicant: RODNEY DEREK LINDSAY, DALLAS, TX (US)

(72) Inventor: RODNEY DEREK LINDSAY, DALLAS, TX (US)

(21) Appl. No.: 16/787,744

(22) Filed: Feb. 11, 2020

(51) Int. Cl. A47G 1/06; A47G 1/02; A61B 5/00; G06K 9/20

(52) U.S. Cl. CPC A47G 1/0622; G06K 9/20; A61B 5/6891; A47G 1/02

(57) ABSTRACT

Embodiments of the present invention disclose a foot mirror assembly having a camera for capturing photos of a foot of a user which provide the user an improved view of the foot for diabetic foot exams. The assembly includes a frame member, a standing member, a mirror coupled with the frame member, a set of lights located on the frame member. Other embodiments include an electronic device coupled with the frame member, the electronic device having a camera. In further embodiments, the electronic device transmits captured photographs to a remote device.

### BACKGROUND OF THE INVENTION

### 1. Field of the Invention

The present application relates to mirrors, and more particularly to foot mirrors for diabetic users.

### 2. Description of Related Art

For a user who is diabetic, daily foot exams of the user is paramount to prevention and management of complications resulting from neuropathy and poor blood circulation. To aid in these foot exams, the user may employ a hand-held mirror to better inspect the plantar side of the foot. However, the use of a hand-held mirror restricts the user to have one hand always holding the hand-held mirror, which can make for treatment and dressing of the foot difficult with only one other free hand. Furthermore, users with poor eyesight may have difficulty viewing the plantar side of the foot through a mirror. Therefore, it is desired to develop a foot mirror that is free standing and provides improved visibility over traditional hand-held mirrors.

### BRIEF SUMMARY OF THE INVENTION

It is an object of the present application to provide a foot mirror assembly for assisting a user to view a foot for a diabetic foot exam, the foot mirror assembly comprising: a frame member; a stand member coupled to the frame member forming an angle between the frame member and the stand member; a mirror coupled to the frame member; and a light coupled to the frame member.

Another object of the present application is to provide a foot mirror assembly having further an electronic device coupled to the frame member, the electronic device having a camera for capturing photographs of the foot, transmitting the captured photographs to a remote device, such as a smart phone, and displaying the captured photographs on the remote device.

Another object of the present application is to provide a foot mirror assembly alternatively having an attachment member configured to attach to the frame member of the foot mirror assembly, the attachment member also configured to receive an electronic device having a camera, such as a smart phone, wherein the electronic device is used to capture photographs of the foot. A user operates a wireless remote that transmits a signal to the electronic device to capture a photograph using the camera.

Ultimately the invention may take many embodiments. In these ways, the present invention overcomes the disadvantages inherent in the prior art.

The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the various purposes of the present design. It is important, therefore, that the claims be regarded as including such equivalent constructions in so far as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a mirror assembly having an embedded camera, in accordance with an embodiment of the present invention;

FIG. 2 is a perspective view of a mirror assembly configured to receive a portable device having a camera, in accordance with an embodiment of the present invention;

FIG. 3 is a functional block diagram illustrating a communication network of the components of FIG. 1 and FIG. 2;

FIG. 4 depicts a block diagram of components of the computing system of FIG. 3.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The assembly and method in accordance with the present invention overcomes one or more of the above-discussed problems associated with mirrors for diabetic foot exams. In particular, the system of the present invention is a foot mirror having a camera for capturing photos of a foot of a user, thereby providing the user an improved view of the foot as well as allowing the user to view the foot without having to hold the mirror.

The system will be understood from the accompanying drawings, taken in conjunction with the accompanying description. Several embodiments of the system may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system of the present application is illustrated in the associated drawings. As used herein, “system” and “assembly” are used interchangeably. It should be noted that the articles “a”, “an”, and “the”, as used in this specification, include plural referents unless the content clearly dictates otherwise. Additional features and functions are illustrated and discussed below.

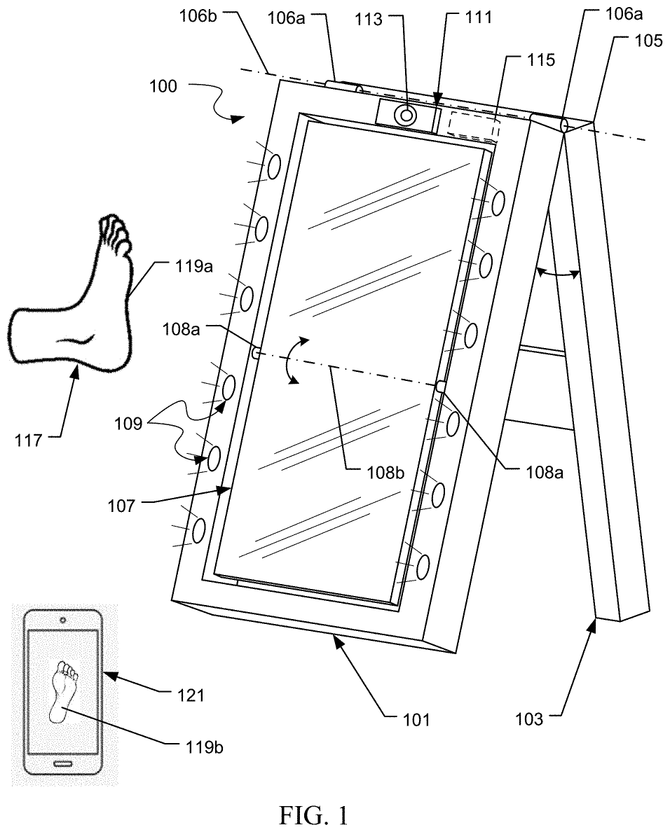

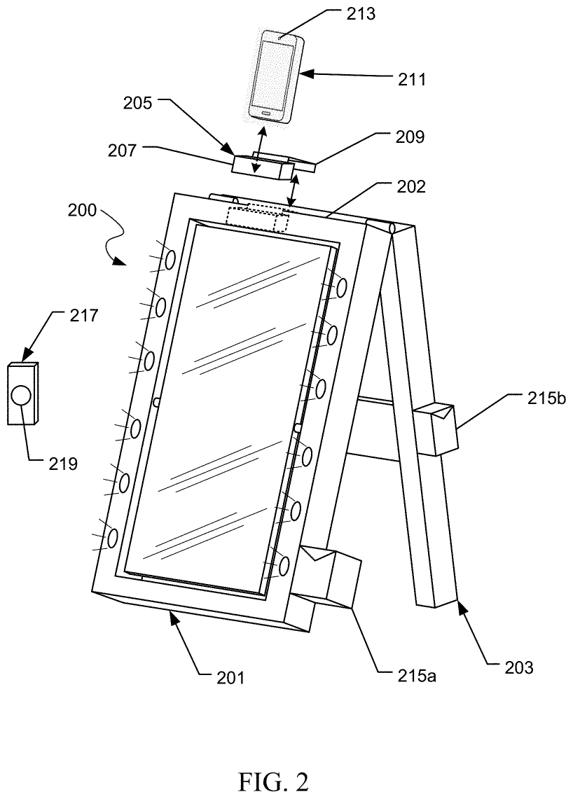

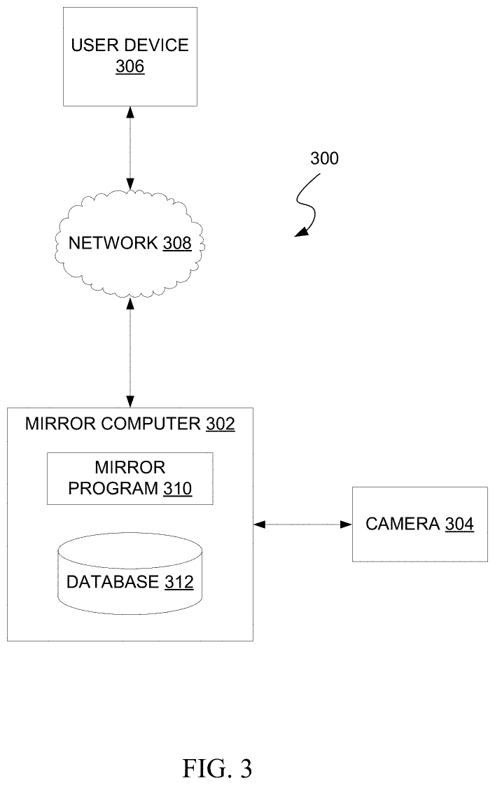

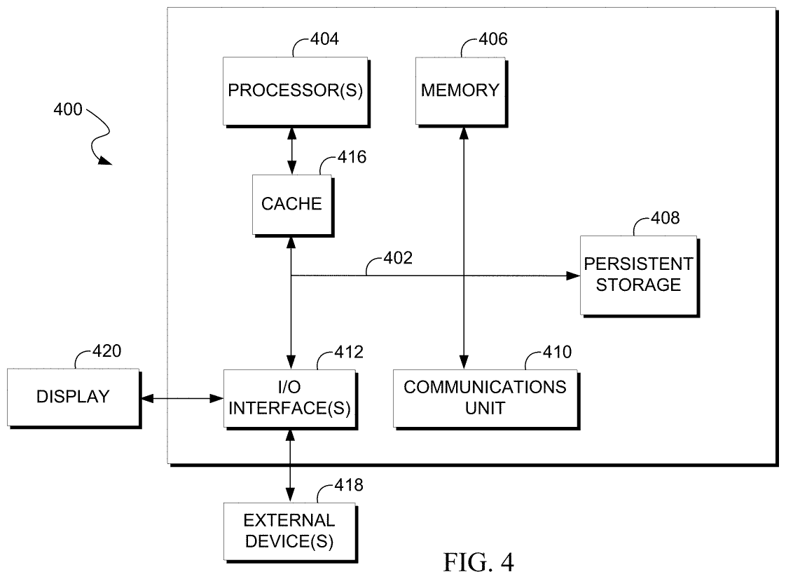

Referring now to the drawings wherein like reference characters identify corresponding or similar elements in form and function throughout the several views. FIGS. 1-2 illustrate perspective views of embodiments of a foot mirror assembly. FIG. 3 illustrates a functional block diagram of a communication network of components of the foot mirror assemblies of FIGS. 1 and 2. FIG. 4 depicts a block diagram of components of the computing system of FIG. 3.

Referring now to FIG. 1, a perspective view of mirror assembly 100 having an embedded camera is illustrated in accordance with an embodiment of the present invention.

In FIG. 1, mirror assembly 100 comprises, but is not limited to, frame member 101, stand member 103, mirror 107, set of lights 109, and electronic device 111. Mirror 107 coupled with frame member 101. For example, in FIG. 1, mirror 107 is framed by frame member 101. Optionally, mirror 107 may be configured to selectively pivot relative to frame member 101 based on a hinge that is coupled between mirror 107 and frame member 101. For example, in FIG. 1, mirror 107 is coupled with frame member 101 based on a hinge 108a that permits mirror 107 to selectively pivot about axis 108b relative to frame member 101. Mirror 107 may be a planar mirror or a concave mirror.

Stand member 103 is a stand for frame member 101. In FIG. 1, stand member 103 is coupled with frame member 101 at end 105 of stand member 103, thus forming an angle between frame member 101 and stand member 103. Optionally, stand member 103 may be configured to selectively pivot relative to frame member 101 based on a hinge that is coupled between frame member 101 and stand member 103 located at end 105. For example, in FIG. 1, stand member 103 is coupled with frame member 101 based on hinge 106a that permits stand member 103 to selectively pivot about axis 106b relative to frame member 101.

A set of lights are one or more lights that assist in illuminating a foot of a user while the foot is held up towards mirror 107. In FIG. 1, set of lights 109 are located on frame member 101.

Electronic device 111 is a computing device having camera 113 that is coupled to frame member 101 for capturing photos of a foot of a user while the foot is held up towards mirror 107 (and subsequently towards camera 113). In general, electronic device 111 can be a device that is permanently coupled to frame member 101 (as depicted in FIG. 1) or a portable device that is selectively detachable from frame member 101 (as further depicted in FIG. 2). In FIG. 1, electronic device 111 is coupled with frame member 101. In this figure, a user holds up plantar side 119a of foot 117 towards mirror 107 and camera 113, and electronic device 111 captures images of plantar side 119a via camera 113. Electronic device 111 in turn transmits the captured images of plantar side and displays the images on a user device. In this figure, electronic device 111 captures images of plantar side 119a and transmits the images to user device 121, wherein user device 121 displays image 119b of plantar side 119a. Components and communication network of electronic device 111 and user device 121 are further depicted in FIG. 3 and FIG. 4 respectively.

Optionally, camera 113 may be configured to receive a filter that enhances visibility of surface terrain of the foot.

In general, components of the mirror assembly (i.e., set of lights 109 and/or electronic device 111) may be powered by conventional means of a home wall outlet; however, alternatively, the mirror assembly may include a power supply located within frame member 101. The power supply may be battery or rechargeable battery. In FIG. 1, power supply 115 is embedded in frame member 101. Optionally, power supply 115 may power a laser in performing laser therapy for wound healing.

As a method of use, a user obtains mirror assembly 100, positions mirror 107 and camera 113 towards the user, positions plantar side 119a of foot 117 corresponding to the user toward mirror 107 and camera 113 wherein plantar side 119a is viewable to the user in mirror 107, and captures a photo of plantar side 119 by transmitting a capture photo signal using a remote device to electronic device 111. While in this example the user positions plantar side 119a of foot 117 towards mirror 107, it should recognized that the user may alternatively position other sides of the foot towards mirror 107 and camera 113.

Referring now to FIG. 2, a perspective view of mirror assembly 200 configured to receive a portable device having a camera is illustrated in accordance with an embodiment of the present invention.

In FIG. 2, mirror assembly 200 is of the same form and function as mirror assembly 100 except for the electronic device being a portable device that is selectively detachable from frame member 101. In other words, frame member 201 is the same as frame member 201, stand member 203 is the same as stand member 103, etc.

In this figure, electronic device 211 has camera 213 and is a portable device (e.g., mobile phone) that is selectively detachable from frame member 201 using an attachment member. In general, an attachment member is any device that is capable of selectively coupling an electronic device to frame member 201. In this figure, attachment member 205 comprises of, but is not limited to, a cradle portion (i.e., cradle 207) and a hook portion (i.e., hook 209), wherein cradle 207 is configured to receive electronic device 211, and hook 209 is configured to receive end 202 of frame member 201. In this setup, a user may capture photos of the foot of the user using electronic device 211 and remote 217 by engaging button 219 on remote 217. In this figure, remote 217 is a wireless remote that is capable of transmitting a signal to electronic device 211, wherein the signal instructs electronic device 211 to capture a photo using camera 213.

Optionally, toolkits may be affixed to the mirror assembly. For example, in FIG. 2, toolkit 215a is coupled to frame member 201, and toolkit 215b is coupled to stand member 203. Toolkits 215a-b are configured to include, but is not limited to, one or more tools such as monofilaments, tweezers, probes, etc.

Referring now to FIG. 3, a functional block diagram illustrating communication network 300 of the components of a mirror assembly. FIG. 3 provides only an illustration of one implementation and does not imply any limitations with regard to the environments in which different embodiments may be implemented. Many modifications to the depicted environment may be made by those skilled in the art without departing from the scope of the invention as recited by the claims.

Communication environment 300 includes mirror computer 302, camera 304, and user device 306, all interconnected over network 308. Mirror computer 302 and user device 306 can be a standalone computing device, a management server, a webserver, a mobile computing device, or any other electronic device or computing system capable of receiving, sending, and processing data. In other embodiments, mirror computer 302 and user device 306 can represent a server computing system utilizing multiple computers as a server system, such as in a cloud computing environment. In another embodiment, mirror computer 302 and user device 306 can be a laptop computer, a tablet computer, a netbook computer, a personal computer (PC), a desktop computer, a personal digital assistant (PDA), a smart phone, or any programmable electronic device capable of communicating with various components and other computing devices (not shown) within communication environment 300. In another embodiment, mirror computer 302 and user device 306 each represent a computing system utilizing clustered computers and components (e.g., database server computers, application server computers, etc.) that act as a single pool of seamless resources when accessed within communication environment 300. Mirror computer 302 and user device 306 may include internal and external hardware components capable of executing machine-readable program instructions for capturing and transmitting images. In some instances, mirror computer 302 and user device 306 may be a single device.

In general, user device 306 is a device that a user operates that sends a signal to mirror computer 302 to capture a photo using camera 304. In general, mirror computer 302 is a device that is coupled (selectively or permanently) to the frame member (i.e., frame member 101 or 201) of the mirror assembly. In some instances, mirror computer 302 may transmit images to user device 306. For example, in light of FIG. 1, user device 306 can be a smart phone or smart tablet (i.e., user device 121) and mirror computer 302 is electronic device 111. In another example, in light of FIG. 2, user device 306 can be remote 217 and mirror computer 302 is a selectively attachable device such as a smart phone or smart tablet (i.e., electronic device 211).

Mirror computer 302 includes mirror program 310 and database 312. In general, mirror program 310 receives signals to capture a photo from user device 306 (i.e., receives a capture photo selection from a user), in response to receiving the signals from user device 306, captures a photo using camera 304, transmits the captured photo to user device 306, and displays the captured photo on a display screen of the user device 306. In some embodiments, mirror program 310 stores the capture photo to database 312.

Database 312 is a repository for data accessible by mirror program 310. Database 312 can be implemented with any type of storage device capable of storing data and configuration files that can be accessed and utilized by server computer 302, such as a database server, a hard disk drive, or a flash memory. Database 312 stores a set of captured images collected from camera 304.

Network 308 can be, for example, a telecommunications network, a local area network (LAN), a wide area network (WAN), such as the Internet, or a combination of the three, and can include wired, wireless, or fiber optic connections. Network 308 can include one or more wired and/or wireless networks that are capable of receiving and transmitting data, voice, and/or video signals, including multimedia signals that include voice, data, and video information. In general, network 308 can be any combination of connections and protocols that will support communications among mirror computer 302 and user device 306 and other computing devices (not shown) within communication environment 300.

FIG. 4 depicts a block diagram of components of computing systems within communication environment 300 of FIG. 3, in accordance with an embodiment of the present invention. It should be appreciated that FIG. 4 provides only an illustration of one implementation and does not imply any limitations with regard to the environments in which different embodiments can be implemented. Many modifications to the depicted environment can be made.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

Computer system 400 includes communications fabric 402, which provides communications between cache 416, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a crossbar switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

Mirror program 310 may be stored in persistent storage 408 and in memory 406 for execution by one or more of the respective computer processors 404 via cache 416. In an embodiment, persistent storage 408 includes a magnetic hard disk drive. Alternatively, or in addition to a magnetic hard disk drive, persistent storage 408 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices. In these examples, communications unit 410 includes one or more network interface cards. Communications unit 410 may provide communications through the use of either or both physical and wireless communications links. Mirror program 310 may be downloaded to persistent storage 408 through communications unit 410.

I/O interface(s) 412 allows for input and output of data with other devices that may be connected to mirror computer 302 and user device 306. For example, I/O interface 412 may provide a connection to external devices 418 such as a keyboard, keypad, a touch screen, a microphone, and/or some other suitable input device. External devices 418 can also include portable computer readable storage media such as, for example, thumb drives, portable optical or magnetic disks, and memory cards. Software and data used to practice embodiments of the present invention, e.g., mirror program 310, can be stored on such portable computer readable storage media and can be loaded onto persistent storage 408 via I/O interface(s) 412. I/O interface(s) 412 also connect to a display 420.

Display 420 provides a mechanism to display data to a user and may be, for example, a computer monitor.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A foot mirror assembly, comprising:
a frame member;
a stand member coupled with the frame member forming an angle between the frame member and the stand member;
a mirror coupled to the frame member; and
a light coupled to the frame member.

2. The system of claim 1, further comprising:
an electronic device coupled to the frame member, the electronic device comprising:
one or more computer processors;
one or more computer readable storage media; and
a camera.

3. The system of claim 2 further comprising a wireless remote that wirelessly transmits a signal to the electronic device, the signal instructing the electronic device to capture a photo using the camera.

4. The system of claim 2, further comprising an attachment member configured to attach to the frame member, the attachment member configured to receive the electronic device, wherein the electronic device is coupled to the frame member via the attachment member.

5. The system of claim 3, wherein the electronic device is a portable device that is selectively detachable from the attachment member.

5. The system of claim 1, wherein the stand member is configured to selectively pivot about an axis relative to the frame member.

6. The system of claim 1, wherein the mirror is configured to selectively pivot about an axis relative to the frame member.

7. The system of claim 1, further comprising a toolkit coupled to at least one of the stand member and the frame member, the toolkit configured to include one or more tools.

8. A method of using a foot mirror assembly, comprising:
obtaining the foot mirror assembly of claim 2;
positioning the camera towards a user;
positioning a foot of the user towards the mirror and the camera, wherein the foot is viewable to the user in the mirror; and
capturing a photo of the foot using the electronic device.

9. The method of claim 8, further comprising positioning the foot mirror assembly by selectively pivoting the stand member about an axis relative to the frame member.

10. The method of claim 8, further comprising positioning the mirror by selectively pivoting the mirror about an axis relative to the frame member.

11. The method of claim 8, wherein capturing a photo of the foot using the electronic device comprises:
in response to receiving a capture photo selection from a remote device, capturing a photo using the camera;
transmitting the captured photo to the remote device; and
displaying the captured photo on the remote device.

12. The method of claim 11, wherein the remote device is a wireless device that wirelessly transmits a signal to the electronic device, the signal instructing the electronic device to capture the photo using the camera.

13. A foot mirror assembly, comprising:
a frame member;
a stand member coupled to the frame member forming an angle between the frame member and the stand member;
a mirror coupled to the frame member;
a light coupled to the frame member;
an electronic device coupled to the frame member, the electronic device comprising:
one or more computer processors;
one or more computer readable storage media;
a camera located on the frame member; and
program instructions stored on the one or more computer readable storage media for execution by at least one of the one or more processors, the program instructions comprising:
in response to receiving a capture photo selection, program instructions to capture a photo using the camera;
program instructions to transmit the captured photo to a remote device; and
program instructions to display the captured photo on the remote device.

14. The system of claim 13, wherein the stand member is configured to selectively pivot about an axis relative to the frame member.

15. The system of claim 13, wherein the mirror is configured to selectively pivot about an axis relative to the frame member.

16. The system of claim 13, wherein the mirror is a planar mirror.

17. The system of claim 13, wherein the mirror is a concave mirror.

18. The system of claim 13, wherein a power supply is stored within the frame member, the power supply powering the light and the electronic device.

19. The system of claim 13, further comprising a wireless remote that wirelessly transmits a signal to the electronic device, the signal instructing the electronic device to capture a photo using the camera.

20. The system of claim 13, further comprising a toolkit coupled to at least one of the stand member and the frame member, the toolkit configured to include one or more tools.
